(19) United States
(12) Patent Application Publication
(10) Pub. No.: US 2025/0064763 A1
(43) Pub. Date: Feb. 27, 2025

(54) METHODS OF TREATMENT OF PRIMARY BILIARY CHOLANGITIS

(71) Applicant: Ipsen Pharma, Boulogne-Billancourt (FR)

(72) Inventors: Benjamin Miller, Cambridge, MA (US); Claudia Zein, Cambridge, MA (US); Jianfen Shu, Cambridge, MA (US); Richard Allan, Wrexham (GB); Nuno Antunes, Cambridge, MA (US)

(21) Appl. No.: 18/780,069

(22) Filed: Jul. 22, 2024

(30) Foreign Application Priority Data
Aug. 22, 2023 (EP) 23192553.8

(51) Int. Cl. A61K 31/192; A61K 45/06

(52) U.S. Cl. CPC A61K 31/192; A61K 45/06

(57) ABSTRACT

The present invention concerns elafibranor for use for the treatment of Primary Biliary Cholangitis (PBC) in a subject with PBC and with liver cirrhosis, and preferably a subject having Child-Pugh score A or having Child-Pugh score B.

### FIELD OF THE INVENTION

The present invention relates to the field of medicine, in particular the treatment of Primary Biliary Cholangitis.

### BACKGROUND OF THE INVENTION

The present invention is dedicated to PBC (Primary Biliary Cholangitis, previously named Primary Biliary Cirrhosis), which is a cholestatic disease.

Cells in the liver produce bile, which passes through ducts within the liver to the gallbladder. Bile is a digestive liquid that is made in the liver. It travels through the bile ducts to the gallbladder and the small intestine, where it helps digest fats and fatty vitamins.

PBC is a chronic inflammatory intrahepatic, or long-term, liver disorder that slowly destroys the small-to-medium-sized bile ducts (tube-like structures that carry bile) within the liver. In patients with PBC, the bile ducts are destroyed by inflammation. This causes bile to remain in the liver, where gradual injury damages liver cells and causes cirrhosis, or scarring of the liver.

PBC is a rare, chronic, progressive autoimmune cholestatic liver disease characterized by lymphocytic cholangitis and associated with increased mortality. In untreated patients or those with an inadequate response to ursodeoxycholic acid (UDCA), PBC commonly progresses to fibrosis, cirrhosis, liver failure and death unless a liver transplant is received.

The estimated prevalence of PBC in North America, Europe, and the Asia Pacific region varies from 1.91 to 40.2 per 100,000 persons and the incidence varies from 0.23 to 5.31 per 100,000 persons, with the incidence increasing over time in North America and Europe. PBC predominantly affects women, with a female to male ratio of approximately 9:1. PBC is typically diagnosed between 40 and 60 years of age, and global estimates suggest that 1 in 1,000 females aged>40 years are living with PBC.

Approximately 60% of patients with PBC are asymptomatic at the time of diagnosis, often having been referred to a hepatologist for abnormal liver enzyme tests (most commonly, elevated alkaline phosphatase (ALP) or gamma glutamyl transferase (GGT) noted at the time of a routine health assessment. The majority of patients become symptomatic within 10 years of diagnosis. Initial presentation in symptomatic patients may include fatigue, pruritus, weight loss, right upper abdominal quadrant pain, and sometimes jaundice. According to practice guidelines, a diagnosis of PBC is based on the presence of at least two of three of the following diagnostic criteria: an elevated serum ALP level (>1.5 times the upper limit of normal [ULN]), histologic evidence of chronic nonsuppurative biliary ductal destruction (florid duct lesion), and presence of anti-mitochondrial antibodies (AMA) in blood at a titre of 1:40 or greater. Liver biopsy is generally not required for diagnosis but can be useful in cases without a clear diagnosis, such as patients with negative AMA, or cases where differential diagnosis for potential clinically similar conditions, such as autoimmune hepatitis and non-alcoholic steatohepatitis (NASH) is needed.

In early PBC, patients are usually asymptomatic despite underlying inflammatory injury of small bile ducts, and slight anomalies in serum liver biochemical tests; this phase may continue for decades. An intermediate phase of PBC follows, where biochemical and clinical symptoms of cholestasis develop, while underlying lesions progress to ductopenia and liver fibrosis; this phase can continue for up to 10 years or more. In advanced PBC, patients may develop progressive jaundice, portal hypertension, and liver failure, sometimes deteriorating over the span of 2 to 4 years and progressing to liver related death in the absence of liver transplant. Hepatocellular carcinoma also may develop in advanced stage PBC.

Nearly all patients with PBC will become symptomatic during the course of their disease. Pruritus and fatigue are the most frequent symptoms. Other common symptoms include sicca complex, abdominal pain, arthralgia, restless legs, sleeplessness, depression and cognitive dysfunction.

Pruritus affects up to 70% of patients with PBC and can contribute to substantial morbidity. Because pruritus follows the circadian rhythm and is often worse at night, patients with PBC may also suffer from diminished sleep quality, leading to increased fatigue and a negatively impacted QoL. Pruritus may occur during early stages of the disease and may persist even in the setting of biochemical response or ALP normalization following treatment with UDCA. Fatigue affects up to 80% of patients with PBC and also negatively affects QoL. Fatigue often interferes with the patient's ability to perform activities of daily living and is associated with diminished mental and physical capacity. While pruritus may improve with liver transplantation, fatigue frequently persists even after liver transplantation.

Several definitions for inadequate response to treatment have been published that incorporate liver biochemical parameters, ALP and bilirubin being considered the most important. According to the latest guidelines, those with an inadequate response to treatment after 12 months of first line therapy using any of the published binary biochemical criteria should be considered for second line therapy due to the increased risk of progressive liver disease; however, a potential limitation of this step up approach to patient management is that those at highest risk of disease progression (i.e., those that do not respond to first line therapy) wait the longest to receive effective treatment. The early addition of second line therapy in patients with an inadequate response to first line therapy becomes especially important when trying to prevent progression of PBC.

The only approved medical therapies in the US and Europe for the treatment of PBC are UDCA, a hydrophilic, noncytotoxic bile acid and obeticholic acid (OCA, Ocaliva®) a semi synthetic analogue of the primary bile acid chenodeoxycholic acid, which selectively activates the nuclear hormone receptor farnesoid X receptor (FXR).

UDCA at a daily dose of 13 to 15 mg/kg/day is the only approved first line therapy for the treatment of PBC. Treatment with UDCA has been shown to improve liver biomarkers of response, slow down the disease progression, and improve transplant free survival. However, up to 40% of UDCA treated patients have an inadequate response, and such patients remain at high risk of disease progression and have reduced transplant free survival rates compared to those classified as UDCA responders. Despite ongoing therapy, 26% to 44% of patients with ALP>1.67×ULN progress to liver transplant or death over 15 years.

Furthermore, treatment with UDCA has not demonstrated an effect on the onset or amelioration of symptoms, including pruritus and fatigue, and it is estimated that between 3% to 5% of patients with PBC are UDCA intolerant.

OCA is the only approved second line therapy for the treatment of PBC. OCA was approved by the FDA in 2016 and by the EMA in 2017, for treatment of PBC in combination with UDCA in patients with an inadequate response to treatment with UDCA, or as monotherapy in patients who are unable to tolerate UDCA. Less than 50% of patients treated with OCA, alone or in combination with UDCA, achieve a biochemical response based on ALP reduction (ALP<1.67×ULN and ≥15% decrease from baseline and normal total bilirubin levels). Therefore, the majority of patients remain at risk of disease progression. Use of OCA has also been shown to exacerbate pruritus. In the phase III study with OCA, events of pruritus were more common in the OCA groups compared to placebo and the severity of pruritus was also higher in the OCA groups compared to placebo. Furthermore, more treatment discontinuations due to pruritus occurred with OCA than with placebo. As a result, initiation of OCA at a starting dosage of 10 mg daily is not recommended due to an increased risk of pruritus, and patients with intolerable pruritus due to OCA may need to titrate the dose down to 5 mg daily. OCA has conditional approval in the US and EU based on a reduction in alkaline phosphatase (ALP). An improvement in survival or disease related symptoms has not been confirmed in a controlled clinical trial.

Fibrates, which are commonly used for the treatment of dyslipidaemia, are used off label for the treatment of chronic cholestatic diseases such as PBC. Bezafibrate is commonly used off label as a second line therapy in Europe, while fenofibrate is the most frequently used fibrate in the US. In a randomised, double blind, placebo controlled study in 100 participants with PBC and an inadequate response to UDCA, 24 months of treatment with bezafibrate led to a complete biochemical response in 31% of the patients treated with bezafibrate compared to 0% treated with placebo (p<0.001).

Currently approved therapies for the treatment of PBC have not demonstrated a beneficial effect on the most common symptoms experienced by patients with PBC, pruritus and fatigue. For pruritus, cholestyramine, rifampicin, naltrexone, and sertraline have been recommended in a stepwise approach according to established guidelines. Off label use of bezafibrate for cholestatic pruritus (due to PBC, primary sclerosing cholangitis [PSC] and secondary sclerosing cholangitis [SSC]) has demonstrated greater reduction in moderate to severe pruritus compared with placebo, while evidence supporting an anti-pruritic effect for fenofibrate is less robust. For fatigue, despite the significant burden, no effective therapies have been described to date.

Despite the availability of approved medical therapies for PBC, there is a clear unmet need for additional therapies given the large percentage of non-responders to UDCA at high risk of progression to end stage liver disease, and the limited therapies available to treat the most common symptoms, including pruritus, that have a major impact on patient QoL.

The need for novel therapeutic options for the management of PBC is still clear and urgent.

Elafibranor (GFT505, IPN60190) is an orally administered dual PPARα, δ agonist being developed by Ipsen for the treatment of PBC in combination with UDCA in adults with an inadequate response to UDCA or as monotherapy in adults unable to tolerate UDCA.

In the Phase 2 clinical study NCT03124108 (GFT505B-216-1), evaluating the efficacy and safety of elafibranor in patients with PBC and inadequate response to UDCA, patients with moderate or severe hepatic impairment (defined as Child-Pugh B/C) or with clinically significant hepatic decompensation could not participate because they met one of the exclusion criteria.

In the Phase 3 clinical study NCT04526665 (GFT505B-319-1, ELATIVE), evaluating the effect of daily oral administration of 80 mg elafibranor on cholestasis (impairment of bile formation and/or bile accumulation) in patients with PBC and inadequate response or intolerance to UCDA, patients with clinically significant hepatic decompensation, including patients with cirrhosis/portal hypertension complications, could not participate because they met one of the exclusion criteria.

Based on post marketing reports of liver injury in PBC patients treated with OCA with either compensated or decompensated cirrhosis, which led to hepatic decompensation and liver failure, regulatory agencies have restricted its use in patients with cirrhosis. The use of OCA is now contraindicated in patients with PBC with decompensated cirrhosis (including Child-Pugh Class B or C) or a prior decompensation event, or compensated cirrhosis with evidence of portal hypertension.

Therefore, a clear unmet need for second line therapy still remains, particularly for subjects with PBC and with liver cirrhosis.

### SUMMARY OF INVENTION

A clinical study has surprisingly shown that the treatment of subjects with elafibranor provides a relevant reduction of biochemical markers in the plasma, demonstrating that this compound is advantageous for the treatment of PBC.

It has been also surprisingly shown that such compound is advantageous in a subject with PBC and with liver cirrhosis, and in particular having a Child-Pugh score A or B.

The present invention relates to elafibranor for use for the treatment of Primary Biliary Cholangitis in a subject with PBC and with liver cirrhosis.

Thus, the present invention relates to elafibranor for use for the treatment of Primary Biliary Cholangitis in a subject with PBC and who has been diagnosed with liver cirrhosis.

Preferably, the subject has Child-Pugh score A or has Child-Pugh score B.

Liver cirrhosis is defined as compensated or decompensated and further classified using the Child-Pugh system which is well known to individuals skilled in the art. Cirrhosis patients are classified on the basis of certain clinical parameters. Child Pugh A patients are compensated and may display minimal obvious symptoms. Patients classified as Child Pugh B or Child Pugh C are decompensated and can exhibit outward symptoms such as ascites.

The Child-Pugh score consists of five clinical features including, ascites, hepatic encephalopathy, albumin, total bilirubin and PT-INR and is used to assess the prognosis of chronic liver disease and cirrhosis. Each component is given a numerical score from 1 to 3 and added to provide total scores ranging from 5 to 15. The higher the score the worse prognosis is for the patient. Patients with a total score of 5-6 are classified as Child Pugh A. Patients with a total score of 7-9 are classified as Child Pugh B. Patients with a total score of 10-15 are the most ill and classified as Child Pugh C.

The Child-Pugh score was originally developed in 1973 to predict surgical outcomes in patients presenting with bleeding esophageal varices. Several studies have shown that Child-Pugh score is an independent prognostic marker in the settings of several diseases including Primary Biliary Cholangitis (PBC). Child-Pugh score can be easily calculated at the bedside.

The components of the scoring system and the point allocations are listed in the table below.

| Measure | 1 point | 2 points | 3 points |
|---|---|---|---|
| Total bilirubin (mg/dl) | <2 | 2-3 | >3 |
| Serum albumin (g/dl) | >3.5 | 2.8-3.5 | <2.8 |
| Prothrombin time, | <4.0 | 4.0-6.0 | >6.0 |
| prolongation (s) | | | |
| Ascites | None | Mild | Moderate to Severe |
| Hepatic | None | Grade I-II (or | Grade III-IV (or |
| encephalopathy | | suppressed with | refractory) |
| | | medication) | |

The predicted 1-year survival based on Child Pugh score is presented in the table below:

| | Class A | Class B | Class C |
|---|---|---|---|
| Total Points | 5-6 | 7-9 | 10-15 |
| 1-year survival | 100% | 80% | 45% |

Another object of the invention is elafibranor for use for preventing liver decompensation in a subject with PBC and with liver cirrhosis.

Another object of the invention is elafibranor for use in delaying (e.g., by at least one month, 6 months, 1 year, or 2 years) the onset of liver decompensation in a subject with PBC and with liver cirrhosis in comparison to the average time to onset of liver decompensation in subjects who are untreated or are treated with UDCA, but not elafibranor.

Another object of the invention is elafibranor for use for delaying (e.g., by at least one month, 6 months, 1 year, or 2 years) or preventing liver decompensation in a subject with PBC and who has been diagnosed with liver cirrhosis.

Another object of the invention is elafibranor for use for preventing portal hypertension in a subject with PBC and with liver cirrhosis.

Another object of the invention is elafibranor for use in delaying (e.g., by at least one month, 6 months, 1 year, or 2 years) portal hypertension in a subject with PBC and with liver cirrhosis in comparison to the average time to onset of portal hypertension in subjects who are untreated or are treated with UDCA, but not elafibranor.

Another object of the invention is elafibranor for use for delaying (e.g., by at least one month, 6 months, 1 year, or 2 years) or preventing portal hypertension in a subject with PBC and who has been diagnosed with liver cirrhosis.

Another object of the invention is elafibranor for use for preventing liver transplantation in a subject with PBC and with liver cirrhosis.

Another object of the invention is elafibranor for use in delaying (e.g., by at least one month, 6 months, 1 year, or 2 years) liver transplantation in a subject with PBC and with liver cirrhosis in comparison to the average time to liver transplantation in subjects who are untreated or are treated with UDCA, but not elafibranor.

Another object of the invention is elafibranor for use reducing the risk of (e.g., by at least 25%, 45%, 60%, or 80%) liver transplantation in a subject with PBC and with liver cirrhosis in comparison to the risk of liver transplantation in subjects who are untreated or are treated with UDCA, but not elafibranor.

Another object of the invention is elafibranor for use for preventing delaying (e.g., by at least one month, 6 months, 1 year, or 2 years), or reducing the risk of (e.g., by at least 25%, 45%, 60%, or 80%) liver transplantation in a subject with PBC and who has been diagnosed with liver cirrhosis.

Another object of the invention is elafibranor for use for preventing, delaying, or reducing the risk of progression to liver cirrhosis in a subject with PBC and without liver cirrhosis.

In a particular embodiment of the invention, elafibranor is administered at a dose varying from 0.01 mg to 1 g per administration, preferentially from 1 mg to 150 mg per administration, more preferably from 70 mg to 130 mg, and more preferably at a dose of 80 mg.

In a particular embodiment, elafibranor is administered once a day.

In another particular embodiment, elafibranor is administered once a day at a dose of 80 mg, preferably in combination with UDCA to a subject with PBC and inadequate response to UDCA.

The present invention also provides the use of elafibranor in the preparation of a pharmaceutical composition, preferably an oral pharmaceutical composition, for treating PBC in a subject with PBC and with liver cirrhosis.

The present invention also provides the use of elafibranor in the preparation of a pharmaceutical composition, preferably an oral pharmaceutical composition, for treating PBC in a subject with PBC and who has been diagnosed with liver cirrhosis.

According to the invention, the pharmaceutical composition may be formulated in the form of injectable suspensions, gels, oils, pills, tablets, suppositories, powders, gel caps, capsules, aerosols or means of galenic forms or devices assuring a prolonged and/or slow release.

According to the present invention, the disclosed method, compound, uses, composition or kit concern the treatment of PBC in a subject with PBC and with liver cirrhosis. They also concern the prevention of liver decompensation in a subject with PBC and with liver cirrhosis, of liver decompensation in a subject with PBC and with liver cirrhosis, of liver transplantation in a subject with PBC and with liver cirrhosis and/or of progression to liver cirrhosis in a subject with PBC and without liver cirrhosis.

According to the present invention, the disclosed method, compound, uses, composition or kit concern the treatment of PBC in a subject with PBC and who has been diagnosed with liver cirrhosis. They also concern the prevention of liver decompensation in a subject with PBC and who has been diagnosed with liver cirrhosis, of liver decompensation in a subject with PBC and who has been diagnosed with liver cirrhosis, of liver transplantation in a subject with PBC and who has been diagnosed with liver cirrhosis and/or of progression to liver cirrhosis in a subject with PBC and who has not been diagnosed with liver cirrhosis.

### Abbreviations and Definitions

In the context of the present invention, the following abbreviations and empirical formulae are used in the figures, in the tables, and in the text:
ALP Alkaline Phosphatase
ALT Alanine Transaminase
AMA Anti-mitochondrial antibody
ASBTi Apical sodium-codependent bile acid transporter inhibitors
AST Aspartate Aminotransferase
C4 Serum 7a-hydroxy-4-cholesten-3-one
CDCA Chenodeoxycholic acid
CPK Creatine phosphokinase
DCA Deoxycholic acid
ECG Electrocardiogram
eGFR Estimated glomerular filtration rate
EMA European Medicines Agency
FDA Food and Drug Administration
FGF19 Fibroblast growth factor 19
FXR Farnesoid X receptor
GGT Gamma glutamyl transferase
HBV Hepatitis B virus
HDL-C High Density Lipoprotein-Cholesterol
HIV Human immunodeficiency virus
IgM Immunoglobulin M
IL-20 Interleukin-20
IVRS Interactive voice response system
IWRS Interactive web response system
LDL-C Low Density Lipoprotein-Cholesterol
MELD Model for End Stage Liver Disease
MRI Magnetic resonance imaging
NASH Nonalcoholic steatohepatitis
NOX NADPH oxidase
OCA Obeticholic acid
PBC Primary Biliary Cholangitis
PK Pharmacokinetics
PPAR Peroxisome proliferator-activated receptor
PSC Primary Sclerosing Cholangitis
PT-INR Prothrombin Time-International Normalized Ratio
QOL Quality of life
SSC Secondary Sclerosing cholangitis
TG TriGlyceride
TGF Transforming growth factor
UDCA Ursodeoxycholic acid
ULN Upper limit of normal
VAS Visual analogue score

Other features, properties and advantages of the invention will emerge more clearly from the description and examples that follow.

### DETAILED DESCRIPTION

As mentioned above, an object of the present invention is elafibranor for use for the treatment of PBC in a subject with PBC and with liver cirrhosis.

According to a particular embodiment, the subject having PBC and liver cirrhosis has Child-Pugh score A or has Child-Pugh score B.

According to a particular embodiment, the subject having PBC and liver cirrhosis has Child-Pugh score A.

According to a particular embodiment, the subject having PBC and liver cirrhosis has not Child-Pugh score B or C.

According to a particular embodiment, the subject having PBC and liver cirrhosis has Child-Pugh score B.

According to a particular embodiment, the subject having PBC and liver cirrhosis has moderate hepatic impairment.

According to a particular embodiment, the subject having PBC and liver cirrhosis has not Child-Pugh score C.

According to a particular embodiment, the subject having PBC and liver cirrhosis is at risk of decompensated cirrhosis.

Another object of the present invention is thus elafibranor for use for the treatment of PBC in a subject with PBC and with liver cirrhosis, and for the prevention of decompensated cirrhosis.

According to a particular embodiment, the subject having PBC and liver cirrhosis is at risk of liver cirrhosis progression.

According to a particular embodiment, the subject having PBC and liver cirrhosis shows clinical signs of portal hypertension.

According to a particular embodiment, the subject having PBC and liver cirrhosis is at risk of portal hypertension.

Another object of the present invention is elafibranor for use for preventing or delaying liver decompensation in a subject with PBC and with liver cirrhosis.

In particular, the subject is at risk of decompensated cirrhosis.

In particular, the subject has Child-Pugh score A or has Child-Pugh score B.

Another object of the present invention is elafibranor for use for preventing or delaying portal hypertension in a subject with PBC and with liver cirrhosis.

In particular, the subject is at risk of portal hypertension.

In particular, the subject has Child-Pugh score A or has Child-Pugh score B.

Another object of the present invention is elafibranor for use for preventing, delaying, or reducing the risk of liver transplantation in a subject with PBC and with liver cirrhosis.

In particular, the subject is at risk of liver transplantation.

In particular, the subject has Child-Pugh score A or has Child-Pugh score B.

Another object of the present invention is elafibranor for use for preventing, delaying, or reducing the risk of progression to liver cirrhosis in a subject with PBC and without liver cirrhosis.

In particular, the subject is at risk of liver cirrhosis.

In a particular embodiment, the subject has PBC and responds at least partly to UDCA.

In another embodiment, the subject has PBC and does not respond adequately to UDCA.

In another embodiment, the subject has PBC and is intolerant to UDCA.

In a particular embodiment, elafibranor is administered, preferably orally, to a subject with PBC and inadequate response to UDCA, in particular at a dose of 80 mg or 120 mg.

The term “an effective amount” or “therapeutic effective amount” refers to an amount of the compound sufficient to produce the desired therapeutic result and elafibranor is administered in amounts that are sufficient to display the desired effect.

In a particular aspect, the desired effect is an improvement in alkaline phosphatase and/or GGT levels signing a reduction in cholestasis. Accordingly, the invention also relates to elafibranor for use in the improvement of ALP and/or GGT levels in a subject with PBC and with liver cirrhosis. Accordingly, the invention also relates to elafibranor for use in the improvement of ALP and/or GGT levels in a subject with PBC and who has been diagnosed with liver cirrhosis.

In particular, elafibranor is administered for lowering the activity of ALP and/or GGT.

In a particular embodiment, elafibranor is administered to a subject with PBC and with liver cirrhosis for normalizing ALP, albumin and/or bilirubin level(s). In a particular embodiment, elafibranor is administered to a subject with PBC and who has been diagnosed with liver cirrhosis for normalizing ALP, albumin and/or bilirubin level(s).

In a particular embodiment, the subject is with PBC and with liver cirrhosis and the treatment results in a level of ALP lower than 1.67×ULN (upper limit of normal) and total bilirubin within normal limit. In a particular embodiment, the subject is with PBC and who has been diagnosed with liver cirrhosis and the treatment results in a level of ALP lower than 1.67×ULN (upper limit of normal) and total bilirubin within normal limit. The reference range of total bilirubin is 0.2-1.2 mg/dL. The reference range of direct bilirubin is 0.1-0.4 mg/dL.

In a particular variant of this embodiment, elafibranor is administered for decreasing ALP level by at least 15%.

In another embodiment, the subject is with PBC and with liver cirrhosis and the treatment results in a level of ALP lower than 2×ULN (upper limit of normal) and total bilirubin within normal limit. In another embodiment, the subject is with PBC and who has been diagnosed with liver cirrhosis and the treatment results in a level of ALP lower than 2×ULN (upper limit of normal) and total bilirubin within normal limit. In a particular variant of this embodiment, elafibranor is administered for decreasing ALP level by at least 40%.

In a particular embodiment, elafibranor is administered to a subject with PBC and with liver cirrhosis, to improve bile acids level such as CDCA, cholic acid, litocholic acid and DCA levels. In a particular embodiment, elafibranor is administered to a subject with PBC and who has been diagnosed with liver cirrhosis, to improve bile acids level such as CDCA, cholic acid, litocholic acid and DCA levels.

In a further embodiment, elafibranor is administered for improving Paris I, Paris II, Toronto I, Toronto II or UK-PBC risk score.

In another embodiment, elafibranor is administered to a subject with PBC and with liver cirrhosis for:
improving AST, GGT, 5′-nucleotidase, total bilirubin, conjugated bilirubin, ALT and albumin levels;
improving lipid parameters;
improving C4 and/or FGF19 levels;
improving IgM levels; and
improving 5D-itch scale, PBC 40 QOL, VAS.

In another embodiment, elafibranor is administered to a subject with PBC and who has been diagnosed with liver cirrhosis for:
improving AST, GGT, 5′-nucleotidase, total bilirubin, conjugated bilirubin, ALT and albumin levels;
improving lipid parameters;
improving C4 and/or FGF19 levels;
improving IgM levels; and
improving 5D-itch scale, PBC 40 QOL, VAS.

In another embodiment, elafibranor is administered to a subject with PBC, having or not having liver cirrhosis, for preventing the following adjudicated clinical outcome events: an all-cause mortality, a liver transplant, a progression to cirrhosis, a liver decompensation, a MELD score≥15, and/or a hepatocellular carcinoma. In another embodiment, elafibranor is administered to a subject with PBC, and who has been diagnosed or who has not been diagnosed with liver cirrhosis, for preventing the following adjudicated clinical outcome events: an all-cause mortality, a liver transplant, a progression to cirrhosis, a liver decompensation, a MELD score≥15, and/or a hepatocellular carcinoma.

The term “treatment” or “treating” refers to therapy, prevention, or prophylaxis of PBC in a subject with PBC and with liver cirrhosis.

The treatment involves the administration of elafibranor (such as via the administration of a pharmaceutical composition comprising elafibranor) to a subject (e.g. a patient) with PBC and with or without liver cirrhosis to prevent, cure, delay, reverse, or slow down the progression of the disease, improving thereby the condition of patients.

The term “subject” refers to a mammal and more particularly a human.

The subjects to be treated according to the invention can be appropriately selected on the basis of several criteria associated with PBC such as previous and/or present drug treatments, associated pathologies, genotype, exposure to risk factors, as well as any other relevant biomarker that can be evaluated by means of any suitable immunological, biochemical, or enzymatic method. The subject to be treated is with PBC and with liver cirrhosis, as characterized as follows:
the presence of at least 2 of the following 3 diagnostic factors:
   (i) history of elevated ALP levels for at least 6 months prior to Day 0 (randomization visit);
   (ii) positive Anti-Mitochondrial Antibodies (AMA) titers (>1/40 on immunofluorescence or M2 positive by enzyme-linked immunosorbent assay (ELISA) or positive PBC-specific antinuclear antibodies;
   (iii) liver biopsy consistent with PBC;
ALP≥1.67×upper limit of normal (ULN);
optionally, taking UDCA for at least 12 months (stable dose for ≥6 months) prior to screening visit.

Elafibranor can have different stable isomeric forms.

Synthesis of elafibranor can for example be carried out as described for compound 29 in WO2004/005233. Elafibranor can be formulated as pharmaceutically acceptable salts, being slightly- or nontoxic salts obtained from organic or inorganic bases or acids of elafibranor. These salts can be obtained during the final purification step of the compound or by incorporating the salt into the previously purified compound. The pharmaceutical compositions comprising elafibranor for the treatment of PBC in a subject with PBC and with liver cirrhosis, can comprise one or several excipients or vehicles, acceptable within a pharmaceutical context (e.g. saline solutions, physiological solutions, isotonic solutions, etc., compatible with pharmaceutical usage and well-known by one of ordinary skill in the art). These compositions can comprise one or several agents or vehicles chosen among dispersants, solubilisers, stabilisers, preservatives, etc.

Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc.

These compositions can be formulated in the form of injectable suspensions, gels, oils, pills, suppositories, powders, gel caps, capsules, aerosols, etc., eventually by means of galenic forms or devices assuring a prolonged and/or slow release. For this kind of formulation, agents such as cellulose, carbonates or starches can advantageously be used.

Elafibranor may be administered in an efficient amount by using a pharmaceutical composition as above defined.

Elafibranor can be administered in different ways and in different forms that allow administering said compounds in a therapeutically effective amount.

Thus, for example, it can be administered in a systematic way, per os, by parenteral route, by inhalation, or by injection, such as for example intravenously, by intra-muscular route, by subcutaneous route, by transdermal route, by intra-arterial route, etc.

Oral administration is the preferential route of administration for pharmaceutical compositions comprising elafibranor for the treatment of PBC in a subject with PBC and with liver cirrhosis.

The frequency and/or dose relative to the administration can be adapted by one of ordinary skill in the art, in function of the patient, the pathology, the form of administration, etc. Typically, elafibranor can be administered for the treatment of PBC in a subject with PBC and with liver cirrhosis at doses varying from 0.01 mg to 1 g per administration, preferentially from 1 mg to 150 mg per administration, and more preferably from 70 mg to 130 mg.

Administration can be performed daily or even several times per day, if necessary.

In a particular embodiment, elafibranor is administered once a day.

In another particular embodiment, elafibranor is administered once a day at a dose of 80 mg or 120 mg, preferably at a dose of 80 mg.

In a particular embodiment, elafibranor is administered throughout the patient's life.

In a particular embodiment, elafibranor is not administered as sole therapeutically active agent.

Thus, in a particular embodiment, the invention also relates to the use of elafibranor for the treatment of PBC in a subject with PBC and with liver cirrhosis, in combination with at least one other therapeutically active agent. Thus, in a particular embodiment, the invention also relates to the use of elafibranor for the treatment of PBC in a subject with PBC and who has been diagnosed with liver cirrhosis, in combination with at least one other therapeutically active agent.

The other active agent may in particular be selected from other anti-cholestatic agents such as UDCA or OCA.

The invention thus also relates to the combination of elafibranor with UDCA or OCA, and preferably with UDCA.

The invention also relates to the combination of elafibranor with an anti-cholestatic agent.

Other anti-cholestatic agents include, without limitation:
apical sodium-codependent bile acid transporter inhibitors (ASBTi);
bile acids;
cathepsin inhibitors;
CCR antagonists;
CD40 inhibitors;
CD80 inhibitors;
Dual NOX (NADPH oxidase) 1 & 4 inhibitors;
Farnesoid X receptor (FXR) agonists;
Fibroblast Growth Factor (FGF) 19 recombinant;
Fractalkine ligand inhibitors;
ileal sodium bile acid cotransporter inhibitors;
Monoclonal antibodies;
PPAR alpha agonists;
PPAR gamma agonists;
PPAR delta agonists;
PPAR alpha/gamma agonists;
PPAR alpha/delta agonists;
PPAR gamma/delta agonists; and
PPAR alpha/gamma/delta agonists or PPARpan agonists.

Illustrative apical sodium-codependent bile acid transporter inhibitors include, without limitation, odevixibat; volixibat; maralixibat formerly SHP-625; linerixibat; elobixibat and CJ-14199.

Illustrative bile acids include, without limitation, obeticholic acid and ursodiol (UDCA).

Illustrative cathepsin inhibitors include, without limitation, VBY-376; VBY-825; VBY-036; VBY-129; VBY-285; Org-219517; LY3000328; RG-7236 and BF/PC-18.

Illustrative CCR antagonists include, without limitation, cenicriviroc (CCR2/5 antagonist); PG-092; RAP-310; INCB-10820; RAP-103; PF-04634817 and CCX-872.

Illustrative CD40 inhibitors include, without limitation, FFp-104; xl-050; DOM-0800; XmAb-5485; KGYY-15; FFP-106; TDI-0028 and ABI-793.

Illustrative CD80 inhibitors include, without limitation, RhuDex; FPT-155; ToleriMab; galiximab; SCH-212394; IGM-001; ASP-2408 and SCH-204698.

Illustrative dual NOX (NADPH oxidase) 1 & 4 inhibitors include, without limitation, GKT-831 (formerly GKT137831) and GKT-901.

Illustrative Farnesoid X receptor (FXR) agonists include, without limitation, obeticholic acid; GS-9674; LJN-452; EDP-305; AKN-083; INT-767; GNF-5120; LY2562175; INV-33; NTX-023-1; EP-024297; Px-103 and SR-45023.

Illustrative Fibroblast Growth Factor 19 (FGF-19) recombinants include, without limitation, NGM-282. Illustrative Fractalkine ligand inhibitors include, without limitation, E-601 1 and KAN-0440567. Illustrative ileal sodium bile acid cotransporter inhibitors include, without limitation, odevixibat; GSK-2330672; volixibat; CJ-14199 and elobixibat.

Illustrative monoclonal antibodies include, without limitation, bertilimumab; NGM-313; IL-20 targeting mAbs; fresolimumab (antiTGF3) formerly GC1008; timolumab formerly BTT-1023; namacizumab; omalizumab; ranibizumab; bevacizumab; lebrikizumab; epratuzumab; felvizumab; matuzumab; monalizumab; reslizumab and inebilizumab.

Illustrative PPAR alpha agonists include, without limitation, fenofibrate, ciprofibrate, pemafibrate, gemfibrozil, clofibrate, binifibrate, clinofibrate, clofibric acid, nicofibrate, pirifibrate, plafibride, ronifibrate, theofibrate, tocofibrate and SR10171.

Illustrative PPAR gamma agonists include, without limitation, Pioglitazone, deuterated pioglitazone, Rosiglitazone, efatutazone, ATx08-001, OMS-405, CHS-131, THR-0921, SER-150-DN, KDT-501, GED-0507-34-Levo, CLC-3001 and ALL-4.

Illustrative PPAR delta agonists include, without limitation, GW501516 (Endurabol or ({4-[({4-methyl-2-[4-(trifluoromethyl)phenyl]-1,3-thiazol-5-yl}methyl) sulfanyl]-2-methylphenoxy}acetic acid)) or MBX8025 (Seladelpar or {2-methyl-4-[5-methyl-2-(4-trifluoromethyl-phenyl)-2H-[1,2,3]triazol-4-ylmethylsylfanyl]-phenoxy}-acetic acid) or GW0742 ([4-[[[2-[3-fluoro-4-(trifluoromethyl)phenyl]-4-methyl-5-thiazolyl]methyl]thio]-2-methyl phenoxy]acetic acid) or L165041 or HPP-593 or NCP-1046.

Illustrative PPAR alpha/gamma agonists (also named glitazars) include, without limitation, saroglitazar, aleglitazar, muraglitazar, tesaglitazar and DSP-8658.

In addition to elafibranor, illustrative PPAR alpha/delta agonists include, without limitation, T913659.

Illustrative PPAR gamma/delta agonists include, without limitation, linoleic acid (CLA) and T3D-959.

Illustrative PPAR alpha/gamma/delta agonists (or “PPARpan agonists”) include, without limitation, IVA337, TTA (tetradecylthioacetic acid), bavachinin, GW4148, GW9135, bezafibrate, lobeglitazone, and CS038. In a further embodiment, the present invention provides methods of treating a subject with PBC and with liver cirrhosis comprising the administration of elafibranor, in particular in the form of a pharmaceutical composition containing this compound, preferably an oral pharmaceutical composition containing this compound. In a further embodiment, the present invention provides methods of treating a subject with PBC and who has been diagnosed with liver cirrhosis comprising the administration of elafibranor, in particular in the form of a pharmaceutical composition containing this compound, preferably an oral pharmaceutical composition containing this compound.

In a further embodiment, the present invention provides methods of treating a subject with PBC and without liver cirrhosis comprising the administration of elafibranor, in particular in the form of a pharmaceutical composition containing this compound, preferably an oral pharmaceutical composition containing this compound.

In another embodiment, the present invention also provides a kit for treating a subject with PBC and with liver cirrhosis comprising elafibranor, optionally in combination to another anti-cholestatic agent as described above.

In another embodiment, the present invention also provides a kit for treating a subject with PBC and without liver cirrhosis comprising elafibranor, optionally in combination to another anti-cholestatic agent as described above.

As previously mentioned, the present invention also provides the use of elafibranor in the preparation of a pharmaceutical composition, preferably an oral pharmaceutical composition, for treating PBC in a subject with PBC and with liver cirrhosis.

The present invention also provides the use of elafibranor in the preparation of a pharmaceutical composition, preferably an oral pharmaceutical composition, for preventing or delaying liver decompensation in a subject with PBC and with liver cirrhosis.

The present invention also provides the use of elafibranor in the preparation of a pharmaceutical composition, preferably an oral pharmaceutical composition, for preventing or delaying portal hypertension in a subject with PBC and with liver cirrhosis.

The present invention also provides the use of elafibranor in the preparation of a pharmaceutical composition, preferably an oral pharmaceutical composition, for preventing liver transplantation in a subject with PBC and with liver cirrhosis.

The present invention also provides the use of elafibranor in the preparation of a pharmaceutical composition, preferably an oral pharmaceutical composition, for preventing, delaying, or reducing the risk of progression to liver cirrhosis in a subject with PBC and without liver cirrhosis.

In a further embodiment, the present invention also discloses the use of elafibranor in the manufacture of a medicament for treating PBC in a subject with PBC and with liver cirrhosis.

In a further embodiment, the present invention also discloses the use of elafibranor in the manufacture of a medicament for preventing or delaying liver decompensation in a subject with PBC and with liver cirrhosis.

In a further embodiment, the present invention also discloses the use of elafibranor in the manufacture of a medicament for preventing or delaying portal hypertension in a subject with PBC and with liver cirrhosis.

In a further embodiment, the present invention also discloses the use of elafibranor in the manufacture of a medicament for preventing, delaying, or reducing the risk of liver transplantation in a subject with PBC and with liver cirrhosis.

In a further embodiment, the present invention also discloses the use of elafibranor in the manufacture of a medicament for preventing, delaying, or reducing the risk of progression to liver cirrhosis in a subject with PBC and without liver cirrhosis.

The present invention further relates to a method for treating PBC in a subject with PBC and with liver cirrhosis, comprising administering a therapeutic effective amount of elafibranor.

The present invention further relates to a method for preventing or delaying liver decompensation in a subject with PBC and with liver cirrhosis, comprising administering a therapeutic effective amount of elafibranor.

The present invention further relates to a method for preventing or delaying portal hypertension in a subject with PBC and with liver cirrhosis, comprising administering a therapeutic effective amount of elafibranor. The present invention further relates to a method for preventing, delaying, or reducing the risk of liver transplantation in a subject with PBC and with liver cirrhosis, comprising administering a therapeutic effective amount of elafibranor.

The present invention further relates to a method for preventing, delaying, or reducing the risk of progression to liver cirrhosis in a subject with PBC and without liver cirrhosis, comprising administering a therapeutic effective amount of elafibranor.

In another embodiment of the invention, the invention also discloses a kit for treating PBC in a subject with PBC and with liver cirrhosis, the kit comprising elafibranor.

In another embodiment of the invention, the invention also discloses a kit for preventing or delaying liver decompensation in a subject with PBC and with liver cirrhosis, the kit comprising elafibranor.

In another embodiment of the invention, the invention also discloses a kit for preventing or delaying portal hypertension in a subject with PBC and with liver cirrhosis, the kit comprising elafibranor.

In another embodiment of the invention, the invention also discloses a kit for preventing, delaying, or reducing the risk of liver transplantation in a subject with PBC and with liver cirrhosis, the kit comprising elafibranor.

In another embodiment of the invention, the invention also discloses a kit for preventing, delaying, or reducing the risk of progression to liver cirrhosis in a subject with PBC and without liver cirrhosis, the kit comprising elafibranor.

The invention is further described with reference to the following, non-limiting, examples.

The present invention will be better understood by referring to the following examples which are provided for illustrative purpose only and should not be interpreted as limiting in any manner the instant invention.

### EXAMPLES

### Example 1: Clinical Trial for PBC

A phase III randomised, parallel-group, double-blind, placebo-controlled, two-arm clinical trial is conducted in patients with PBC and inadequate response or intolerance to ursodeoxycholic acid to evaluate the efficacy and safety of treatment with elafibranor given orally (80 mg daily) on long term clinical outcomes (approximately 84 months).

Per protocol, participants with and without cirrhosis will be eligible to enrol. The study will aim to enrol at least 30% of participants with cirrhosis, and of the 30% cirrhotics, half (maximum of 15% of the total study population) will be Child Pugh B. In addition, the proportion of participants with LSM <8 kPa will be capped at 30% of the total study population.

### Primary Objectives

The primary objective is to evaluate the efficacy of daily oral administration of elafibranor 80 mg compared to placebo based on time to the occurrence of clinical outcome events in adult participants with PBC.

### Secondary Objectives

The secondary objectives are:
To assess the safety and tolerability of daily long-term oral administration of elafibranor 80 mg compared to placebo in adult participants with PBC.
To evaluate the efficacy of daily oral administration of elafibranor 80 mg compared to placebo in adult participants with PBC on efficacy measures, including:
   Biochemical and clinical markers of response,
   Disease-related symptoms,
   PROs including other disease-related symptoms, and QOL,
   Individual components of the composite primary endpoint and liver-related mortality.
To evaluate the PK of elafibranor and its metabolite GFT1007 in adult participants with PBC using a Population PK approach, including identification of covariates impacting PK variability.

### Inclusion Criteria

1. Must have provided written informed consent (IC)

2. Males or females≥18 years of age

3. Participants with a definite or probable diagnosis of primary biliary cholangitis (PBC) as demonstrated by the presence of ≥2 of the following 3 diagnostic criteria:
i) History of elevated alkaline liver phosphatase (ALP) levels for >6 months prior to screening visit 1 (SV1).
ii) Positive antimitochondrial antibodies (AMA) titres (>1/40 on immunofluorescence or M2 positive by enzyme-linked immunosorbent assay (ELISA)) or positive PBC-specific antinuclear antibodies.
iii) Liver biopsy consistent with PBC.

4. ALP≥1.67×ULN with variability≤40% based on two consecutive values obtained during screening. The interval between the two measurements should be at least 2 weeks (up to 4 weeks).

5. If the mean value of both ALP values is ≥1.67×ULN and the variability between values is ≤40%, the participant is eligible (even if one of the two values is <1.67×ULN).

6. In cases where one of the two values is >1.67×ULN, but the mean value is <1.67×ULN, or if the variability between values is >40%, an additional value during screening may be obtained at least 2 weeks (and up to 4 weeks) after the previous measurement during screening.

7. In cases where an additional value is checked, the participant will be eligible if the variability between the second and the third value is $40%, and the mean value of all ALP values during screening is ≥1.67× ULN.

8. All ALP values during screening should be analyzed via the central laboratory.

9. In cases of ineligibility, the candidate may be subsequently rescreened once at investigator's discretion, and enrolled if stable qualifying values are demonstrated.

10. The baseline value will be the average of all values obtained during screening plus the baseline visit measurement (obtained prior to treatment initiation); but the eligibility of the participant will be based on the screening values.

11. Participants taking UDCA for at least 12 months (at a stable dose for ≥3 months) prior to screening period and expected to remain on stable dose during the study, or unable to tolerate UDCA treatment (no UDCA for ≥3 months) prior to screening period (per country standard-of-care dosing).

12. Participants taking medications for management of pruritus (e.g. cholestyramine, rifampin, naltrexone, sertraline or colchicine) must be on a stable dose for ≥3 months prior to screening period.

13. Contraceptive use by men or women should be consistent with local regulations regarding the methods of contraception for those participating in clinical studies.

Male participants: Male participants are eligible to participate if they agree to the following during the study intervention period and for at least 1 month after the last dose of study intervention:
Refrain from donating sperm.

### Plus Either:

Be abstinent from heterosexual intercourse as their preferred and usual lifestyle (abstinent on a long-term and persistent basis) and agree to remain abstinent.

### OR

Must agree to use contraception/barrier as detailed below:
Agree to use a male condom and should also be advised of the benefit for a female partner to use a highly effective method of contraception as a condom may break or leak as there remains a risk to pregnant women and women of childbearing potential.

Female participants: A female participant is eligible to participate if she is not pregnant or breastfeeding, and one of the following conditions applies:
Is a woman of non-childbearing potential (WONCBP).

### OR

Is a woman of childbearing potential (WOCBP) and using a contraceptive method that is highly effective (with a failure rate of <1% per year), preferably with low user dependency, during the study intervention period and for at least 1 month after the last dose of study intervention. The investigator should evaluate the potential for contraceptive method failure (e.g. noncompliance, recently initiated) in relationship to the first dose of study intervention.
A WOCBP must have a negative highly sensitive pregnancy test (urine or serum, as required by local regulations) within 24 hours before the first dose of study intervention.
If a urine test cannot be confirmed as negative (e.g. an ambiguous result), a serum pregnancy test is required. In such cases, the participant must be excluded from participation if the serum pregnancy result is positive.

14. Capable of giving signed informed consent which includes compliance with the requirements and restrictions listed in the informed consent form (ICF) and in this protocol.

### Exclusion Criteria:

1. History or presence of other concomitant liver disease including but not limited to:
i) Primary sclerosing cholangitis (PSC).
ii) Autoimmune hepatitis (AIH) by simplified Diagnostic Criteria of the International Autoimmune Hepatitis Group (IAIHG)≥6, or if treated for an overlap of PBC with AIH, or if there is clinical suspicion and evidence of overlap AIH features, that cannot be explained alone by insufficient response to UDCA.
iii) Positive hepatitis B surface antigen (HBsAg). Participants with negative HBsAg and positive hepatitis B core antibody (HBcAb) may be eligible if hepatitis B virus deoxyribonucleic acid (HBV DNA) is negative.
iv) Hepatitis C virus (HCV) infection defined by positive anti-HCV antibody and positive HCV ribonucleic acid (RNA) (Note: Participants with positive anti-HCV antibody due to previously treated HCV infection, may be enrolled if a confirmatory HCV RNA is undetectable and sustained viral response has been documented).
v) Alcohol-associated liver disease (ALD).
vi) Nonalcoholic steatohepatitis (NASH).
vii) Other chronic liver diseases, such as alpha-1 antitrypsin deficiency.

2. Participants with known cirrhosis who have a Child Pugh C classification. Participants with cirrhosis with Child Pugh A or B classification are allowed.

The inclusion of Child Pugh A and Child Pugh B participants will enrich the population of the study such that it will enable the study of elafibranor in a broader range of PBC patients, ensuring the inclusion of those with more advanced/severe disease. The inclusion of these participants will ensure that the safety and efficacy of elafibranor is confirmed across the broad range of PBC patients that might ultimately be treated with elafibranor. The regular (every 6 months) clinic visits, including ultrasounds and liver stiffness assessment and 3 monthly visits including laboratory safety tests and vital signs will ensure the close oversight and assessment of all participants in the study.

3. History or presence of clinically significant hepatic decompensation, including:
i) History of liver transplantation, current placement on a liver transplant list, current model for end-stage liver disease including serum sodium (MELD)-Na score≥12 due to hepatic impairment (MELD-Na will be calculated only when MELD>11).
ii) Evidence of complications of cirrhosis, including hepatic decompensation or evidence of significant portal hypertension complications including presence of uncontrolled ascites; history of variceal bleeding or related interventions (e.g. variceal banding, or transjugular intrahepatic portosystemic shunt placement); presence of hepatic encephalopathy Grade 2 or higher per West Haven criteria; history or presence of spontaneous bacterial peritonitis. Note: participants with low-risk varices (Grade I) without history of bleeding or other treatment may be eligible to enrol. For Child Pugh B participants (i.e., before the cap is met), hepatic encephalopathy up to Grade 2, controlled ascites on stable diuretic therapy, or known oesophageal varices up to Grade 2 with no bleeding stigmata, may be eligible as long as all other eligibility criteria are met, including the Child Pugh B score.
iii) Hepatorenal syndrome (HRS) (type I or II).

4. Known history of human immunodeficiency virus (HIV) infection or having a positive confirmatory test for HIV type 1 or 2.

5. Medical conditions that may cause non-hepatic increases in ALP (e.g. Paget's disease).

6. Evidence of any other unstable or untreated clinically significant immunological, endocrine, haematologic, gastrointestinal, neurological, or psychiatric disease as evaluated by the investigator; other clinically significant conditions that are not well controlled.

7. Non-hepatic medical conditions that may diminish life expectancy to <2 years, including known cancers.

8. History of hepatocellular carcinoma.

9. Alpha-fetoprotein (AFP)>20 ng/ml with 4-phase liver computerized tomography (CT) or magnetic resonance imaging (MRI) imaging suggesting presence of hepatocellular carcinoma.

10. Known malignancy or history of malignancy within the last 5 years, with the exception of local, successfully treated basal cell carcinoma or in-situ carcinoma of the uterine cervix.

11. Administration of the following medications is prohibited during the study, and prior to the study as per the timelines specified below:
3 months prior to screening period: fibrates, seladelpar, glitazones, obeticholic acid, azathioprine, cyclosporine (systemic), methotrexate, mycophenolate, pentoxifylline, budesonide and other systemic corticosteroids (parenteral and oral chronic administration only); potentially hepatotoxic drugs (including α-methyl-dopa, sodium valproic acid, isoniazid or nitrofurantoin).

12. Participants with previous exposure to elafibranor.

13. Participants who are currently participating in, plan to participate in, or have participated in an investigational drug study or medical device study containing active substance within 30 days or 5 half-lives, whichever is longer, prior to the screening period.
i) If the previous study was for an experimental therapy being studied for potential benefit in PBC, and the potential therapeutic agent was proven to have no beneficial effect in PBC and there are no safety concerns, the participant may enrol after 30 days or 5 half lives from the last dose of the therapeutic agent, whichever is longer.
ii) For therapeutic agents being studied for potential benefit in PBC for which it is still unclear if there may be a potential benefit, participants may enrol after 6 months from the last dose of the therapeutic agent.

14. Electrocardiogram (ECG) with QT interval corrected by Fridericia's formula (QTcF)>450 msec in males or QTcF>470 msec in females for participants without bundle branch block. For participants with bundle branch block or other intraventricular conduction delay, a longer QTcF>480 msec would be exclusionary.

15. Total bilirubin (TB)>3×ULN. Participants with Gilbert's syndrome are eligible with a total bilirubin above 3×ULN if direct bilirubin is <30% of total bilirubin.

16. Alanine aminotransferase (ALT) and/or aspartate aminotransferase (AST)>5×ULN at SV1, or variability>40% based on two consecutive values. The interval between the two measurements should be of at least 2 weeks (and up to 4 weeks):
For ALT and/or AST, if both measurements during screening are ≤5×ULN and the variability between values is ≤40%, the participant is eligible.
For ALT and/or AST, in cases where one of the two values is >5×ULN, or if the variability between values is >40%, an additional value during screening may be obtained at least 2 weeks (and up to 4 weeks) after the previous measurement. If the repeat value is <5×ULN, the average of all values during screening is <5×ULN, and the variability from the previous value is $40%, the participant may be eligible.
For AST and/or ALT, if both values are <1.5×ULN, there is no limit to the variability between values for eligibility.
In cases of ineligibility, the candidate may be subsequently rescreened at investigator's discretion, and enrolled if stable qualifying values are demonstrated.
All AST and ALT values during screening should be analyzed via the central laboratory.
For both ALT and AST, the baseline value for the purposes of monitoring for DILI will be the average of all values obtained during screening plus the baseline visit measurement (obtained prior to treatment initiation) but the eligibility of the participant will be based on the screening values.

17.

$$Creatinine phosphokinase ( CPK ) > 2 \times ULN .$$

18. Platelet count<75,000/μL.

19. International normalised ratio (INR)>1.5 in the absence of anticoagulant therapy.

20. Estimated glomerular filtration rate (eGFR)<45 mL/min/1.73 m² per the Modification of Diet in Renal Disease (MDRD)-6 Study formula at SV1. In cases of decreased eGFR where the investigator believes the value may not be representative of the actual potential participant's eGFR, retest after adequate hydration is allowed.

21. Significant renal disease, including nephritic syndrome, chronic kidney disease (CKD) (defined as participants with evidence of significantly impaired kidney function or underlying kidney injury).

22. For female participants: known current pregnancy, or has a positive serum pregnancy test, or is breastfeeding.

23. Regular alcohol intake in excess of the recommended limit of 1 standard drink per day for men or women [Crabb 2020 AASLD Practice Guidance], where one standard measure corresponds to 10 g of alcohol.

24. History of alcohol abuse, or other substance abuse within 1 year prior to SV1.

25. A positive drug screen at screening would be exclusionary unless it can be explained by a prescribed medication. Use of cannabidiol (CBD) or other cannabinoids is not exclusionary for this study.

26. Known hypersensitivity to elafibranor or to any of the excipients of the investigational product(s).

27. Mental instability or incompetence, such that the validity of informed consent or ability to be compliant with the study is uncertain.

### Randomization

Patients who satisfy all eligibility criteria will be randomized in a 2:1 ratio to one of the following groups:
Elafibranor 80 mg/day; or
Placebo.

The randomisation ratio is stratified based on the cirrhosis status at baseline and Child Pugh score at baseline, where the strata are defined in the following way: participants with no cirrhosis at baseline, participants with cirrhosis at baseline who are not Child Pugh B, and participants with cirrhosis who are Child Pugh B. The study enrolls at least 30% of participants with cirrhosis, and of the 30% cirrhotics, half (maximum 15% of the total study population) is Child Pugh B. In addition, the proportion of participants with liver stiffness measurement (LSM)<8 kPa at baseline is capped at 30% of the total study population. A central randomization system will be used (interactive voice/web response system [IVRS/IWRS]).

### Primary Endpoint

The primary endpoint is event-free survival, defined as the time from randomisation to the first occurrence of any of the following adjudicated clinical outcome events:
All-cause mortality.
Liver transplant.
Progression to cirrhosis (for those without cirrhosis at baseline), defined as at least one of the following:
   Histologic presence of cirrhosis on liver biopsy performed as part of clinical care;
   LSM>16.9 kPa assessed by VCTE using Fibroscan® in participants with baseline LSM<15 kPa.
Progression to clinically significant portal hypertension, defined as at least one of the following:
   New development of splenomegaly (spleen axis≥13 cm in the largest axis), combined with platelet count<150,000/μL, with platelet count confirmed on repeat testing performed at least 8 weeks apart [EASL Guidelines 2021];
   Development of new oesophageal or gastric varices on EGD, performed as part of clinical care, in participants who did not have varices at baseline (as documented on EGD performed as part of standard clinical care within 12 months prior to screening).
   Evidence on imaging of new portosystemic collaterals.
Liver decompensation, defined as new onset or recurrence of any of the following:
   Variceal bleed;
   Hepatic encephalopathy grade≥2 per West Haven criteria;
   New ascites requiring treatment;
   Refractory ascites or requirement for paracentesis;
   Hepatic hydrothorax;
   Spontaneous bacterial peritonitis;
Hepatorenal syndrome;
Hepatopulmonary syndrome;
Portopulmonary hypertension;
Change in MELD-Na score to ≥15 in participants with baseline MELD or MELD-Na score<12, and in whom MELD score change is reflective of worsening liver disease;
Development of hepatocellular carcinoma.

### Secondary Endpoint

The secondary endpoints are:
to assess at Month 6, Month 12, and every 12 months up to EOT:
   Change from baseline in ALP;
   Change from baseline in TB;
   Number and percentage of participants with ALP≤1.67×ULN and TB≤ULN;
   Number and percentage of participants with normalisation of TB and ALP, defined as TB≤ULN and ALP<ULN;
   Number and percentage of participants with complete biochemical response, defined as normal levels of TB, ALP, transaminases, albumin, and INR;
   Number and percentage of participants with stabilisation in TB (i.e. no increase) defined as TB<1×ULN or increase from baseline<0.1×ULN;
   Number and percentage of participants with a response based on albumin normalization.
to assess at Month 12, and every 12 months up to EOT:
   Change from baseline in LSM assessed by VCTE using Fibroscan®;
   Change from baseline in PBC risk scores: UK PBC score and GLOBE score;
   Number and percentage of participants with LSM≥15 kPa assessed by VCTE using Fibroscan®;
   Number and percentage of participants with no worsening of LSM assessed by VCTE using Fibroscan® defined as no increase of >2 kPa from baseline;
   Change from baseline in AST, ALT, GGT, conjugated bilirubin, albumin, INR and fractionated ALP (hepatic fraction).
to assess at Month 6, Month 12, and every 12 months up to EOT:
   Change from baseline in ALP;
   Change from baseline in TB;
   Number and percentage of participants with ALP≤1.67×ULN and TB≤ULN;
   Number and percentage of participants with normalisation of TB and ALP, defined as TB<ULN and ALP<ULN;
   Number and percentage of participants with complete biochemical response, defined as normal levels of TB, ALP, transaminases, albumin, and INR;
   Number and percentage of participants with stabilisation in TB (i.e. no increase) defined as TB<1×ULN or increase from baseline<0.1×ULN;
   Number and percentage of participants with a response based on albumin normalization.
to assess at Month 12, and every 12 months up to EOT:
   Change from baseline in LSM assessed by VCTE using Fibroscan®;
   Change from baseline in PBC risk scores: UK PBC score and GLOBE score;
   Number and percentage of participants with LSM≥15 kPa assessed by VCTE using Fibroscan®
   Number and percentage of participants with no worsening of LSM assessed by VCTE using Fibroscan® defined as no increase of >2 kPa from baseline;
   Change from baseline in AST, ALT, GGT, conjugated bilirubin, albumin, INR and fractionated ALP (hepatic fraction).
to assess at Month 6, Month 12, and every 12 months up to EOT, as measured by number and percentage of participants with:
   ALP reduction of 40%;
   ALP<1.5×ULN, ALP decrease≥15% and TB≤ULN;
   ALP<1.5×ULN, ALP decrease≥40% and TB≤ULN;
   ALP<1.67×ULN, ALP decrease≥15% and TB≤ULN;
   ALP<3×ULN, AST<2×ULN and TB≤1 mg/dl (Paris I);
   ALP≤1.5×ULN, AST≤1.5×ULN and TB≤1 mg/dL (Paris II criteria);
   Normalisation of abnormal TB;
   Normalisation of abnormal TB and albumin (Rotterdam criteria);
   Reduction in TB to ≤0.6×ULN in participants with TB>0.6×ULN at baseline.
to assess at Month 6, Month 12, and every 12 months up to EOT: Change from baseline in lipid parameters as measured by TC, LDL-C, HDL-C, calculated VLDL-C and TG.
to assess through 6 months up to EOT:
   Change from baseline in PBC Worst Itch NRS score;
   Number and percentage of participants with a response in PBC Worst Itch NRS score defined as ≥2-point reduction from baseline NRS in participants with a baseline NRS≥4;
   Number and percentage of participants with a response in PBC Worst Itch NRS defined as ≥3-point reduction from baseline NRS in participants with a baseline NRS≥4.
to assess at every 6 months up to EOT:
   Change from baseline in 5D-Itch scale;
   Change from baseline in PGI-S;
   Change from baseline in PGI-C;
   Change from baseline in PROMIS Fatigue-Short Form 7a;
   Change from baseline in the ESS;
   Change from baseline in PBC-40 score;
   Change from baseline in EQ-5D-5L;
   Change from baseline in WPAI: GH.
time from randomisation to the first occurrence of each of the following individual adjudicated clinical outcome events:
   All-cause mortality;
   Liver-related mortality;
   Liver transplantation;
   Progression to cirrhosis;
   Progression to clinically significant portal hypertension;
   MELD-Na score≥15 in participants with baseline MELD or MELD-Na score<12;
   Liver decompensation;
   Occurrence of hepatocellular carcinoma;
   Individual PK parameters (during a dosing period of 24 hours) at steady state:
      AUC0-24 (area under the plasma concentration-time curve from time 0 to 24 hours);
      Cmax (maximum (peak) plasma drug concentration);
      tmax (time to reach maximum (peak) plasma concentration following drug administration);
   Population PK parameters:
      CL (apparent clearance of drug from plasma);
      VZ (apparent volume of distribution).

It is expected that elafibranor is better than placebo in preventing clinical outcome events showing disease worsening (including progression of disease leading to liver transplant or death), in participants with PBC and with liver cirrhosis, and more particularly having Child-Pugh score A or having Child-Pugh score B.

### Example 2: Clinical Trial for PBC-Patients with Cirrhosis

A phase III randomised, parallel-group, double-blind, placebo-controlled, two-arm clinical trial is conducted in patients with PBC and cirrhosis and inadequate response or intolerance to ursodeoxycholic acid to evaluate the efficacy and safety of treatment with elafibranor given orally (80 mg daily) on long term clinical outcomes (approximately 42 months).

Per protocol, participants with cirrhosis and Child Pugh A or Child Pugh B will be eligible to enrol. In addition, the proportion of participants with LSM<8 kPa will be capped at 30% of the total study population.

### Primary Objectives

The primary objective is to evaluate the efficacy of daily oral administration of elafibranor 80 mg compared to placebo based on time to the occurrence of clinical outcome events in adult participants with PBC and cirrhosis.

### Secondary Objectives

### The Secondary Objectives are:

To assess the safety and tolerability of daily long-term oral administration of elafibranor 80 mg compared to placebo in adult participants with PBC and cirrhosis.
To evaluate the efficacy of daily oral administration of elafibranor 80 mg compared to placebo in adult participants with PBC and cirrhosis on efficacy measures, including:
   Biochemical and clinical markers of response,
   Disease-related symptoms,
   PROs including other disease-related symptoms, and QOL,
   Individual components of the composite primary endpoint and liver-related mortality.
To evaluate the PK of elafibranor and its metabolite GFT1007 in adult participants with PBC and cirrhosis using a Population PK approach, including identification of covariates impacting PK variability.

### Inclusion Criteria

1. Must have provided written informed consent (IC)

2. Males or females≥18 years of age

3. Participants with a definite or probable diagnosis of primary biliary cholangitis (PBC) as demonstrated by the presence of ≥2 of the following 3 diagnostic criteria:
i) History of elevated alkaline liver phosphatase (ALP) levels for ≥6 months prior to screening visit 1 (SV1).
ii) Positive antimitochondrial antibodies (AMA) titres (>1/40 on immunofluorescence or M2 positive by enzyme-linked immunosorbent assay (ELISA)) or positive PBC-specific antinuclear antibodies.
iii) Liver biopsy consistent with PBC.

4. Participants with cirrhosis at SV1.

5. Participants must be Child Pugh A or Child Pugh B.

6. Participants taking UDCA for at least 12 months (at a stable dose for ≥3 months) prior to screening period and expected to remain on stable dose during the study, or unable to tolerate UDCA treatment (no UDCA for ≥3 months) prior to screening period (per country standard-of-care dosing).

7. Contraceptive use by men or women should be consistent with local regulations regarding the methods of contraception for those participating in clinical studies.

Male participants: Male participants are eligible to participate if they agree to the following during the study intervention period and for at least 1 month after the last dose of study intervention:
Refrain from donating sperm.

### Plus Either:

Be abstinent from heterosexual intercourse as their preferred and usual lifestyle (abstinent on a long-term and persistent basis) and agree to remain abstinent.

### OR

Must agree to use contraception/barrier as detailed below:
Agree to use a male condom and should also be advised of the benefit for a female partner to use a highly effective method of contraception as a condom may break or leak as there remains a risk to pregnant women and women of childbearing potential.

Female participants: A female participant is eligible to participate if she is not pregnant or breastfeeding, and one of the following conditions applies:
Is a woman of non-childbearing potential (WONCBP).

### OR

Is a woman of childbearing potential (WOCBP) and using a contraceptive method that is highly effective (with a failure rate of <1% per year), preferably with low user dependency, during the study intervention period and for at least 1 month after the last dose of study intervention. The investigator should evaluate the potential for contraceptive method failure (e.g. noncompliance, recently initiated) in relationship to the first dose of study intervention.
A WOCBP must have a negative highly sensitive pregnancy test (urine or serum, as required by local regulations) within 24 hours before the first dose of study intervention.
If a urine test cannot be confirmed as negative (e.g. an ambiguous result), a serum pregnancy test is required. In such cases, the participant must be excluded from participation if the serum pregnancy result is positive.

8. Capable of giving signed informed consent which includes compliance with the requirements and restrictions listed in the informed consent form (ICF) and in this protocol.

### Exclusion Criteria:

1. History or presence of other concomitant liver disease including but not limited to:
i) Primary sclerosing cholangitis (PSC).
ii) Autoimmune hepatitis (AIH) by simplified Diagnostic Criteria of the International Autoimmune Hepatitis Group (IAIHG)≥6, or if treated for an overlap of PBC with AIH, or if there is clinical suspicion and evidence of overlap AIH features, that cannot be explained alone by insufficient response to UDCA.
iii) Positive hepatitis B surface antigen (HBsAg). Participants with negative HBsAg and positive hepatitis B core antibody (HBcAb) may be eligible if hepatitis B virus deoxyribonucleic acid (HBV DNA) is negative.
iv) Hepatitis C virus (HCV) infection defined by positive anti-HCV antibody and positive HCV ribonucleic acid (RNA) (Note: Participants with positive anti-HCV antibody due to previously treated HCV infection, may be enrolled if a confirmatory HCV RNA is undetectable and sustained viral response has been documented).
v) Alcohol-associated liver disease (ALD).
vi) Nonalcoholic steatohepatitis (NASH).
vii) Other chronic liver diseases, such as alpha-1 antitrypsin deficiency.

2. Participants with known cirrhosis who have a Child Pugh C classification. Participants with cirrhosis with Child Pugh A or B classification are allowed.

3. History or presence of clinically significant hepatic decompensation, including:
i) History of liver transplantation, current placement on a liver transplant list, current model for end-stage liver disease including serum sodium (MELD) 3.0 score≥12 due to hepatic impairment.
ii) Evidence of complications of cirrhosis, including hepatic decompensation or evidence of significant portal hypertension complications including presence of uncontrolled ascites; history of variceal bleeding or related interventions (e.g. variceal banding, or transjugular intrahepatic portosystemic shunt placement); history or presence of spontaneous bacterial peritonitis. Hepatic encephalopathy up to Grade 2 per West Haven criteria, low-risk varices (Grade I) without history of bleeding, controlled ascites on stable diuretic therapy, or known oesophageal varices up to Grade 2 with no bleeding stigmata, may be eligible as long as all other eligibility criteria are met, including the Child Pugh score.
iii) Hepatorenal syndrome (HRS) (type I or II).
iv) Hospitalisation for liver-related complication within 12 weeks prior to SV1.

4. Known history of human immunodeficiency virus (HIV) infection or having a positive confirmatory test for HIV type 1 or 2.

5. Medical conditions that may cause non-hepatic increases in ALP (e.g. Paget's disease).

6. Evidence of any other unstable or untreated clinically significant immunological, endocrine, haematologic, gastrointestinal, neurological, or psychiatric disease as evaluated by the investigator; other clinically significant conditions that are not well controlled.

7. Non-hepatic medical conditions that may diminish life expectancy to <2 years, including known cancers.

8. History of hepatocellular carcinoma.

9. Alpha-fetoprotein (AFP)>20 ng/ml with 4-phase liver computerized tomography (CT) or magnetic resonance imaging (MRI) imaging suggesting presence of hepatocellular carcinoma.

10. Known malignancy or history of malignancy within the last 5 years, with the exception of local, successfully treated basal cell carcinoma or in-situ carcinoma of the uterine cervix.

11. Administration of the following medications is prohibited during the study, and prior to the study as per the timelines specified below:
3 months prior to screening period: fibrates, seladelpar, glitazones, obeticholic acid, azathioprine, cyclosporine (systemic), methotrexate, mycophenolate, pentoxifylline, budesonide and other systemic corticosteroids (parenteral and oral chronic administration only); potentially hepatotoxic drugs (including α-methyl-dopa, sodium valproic acid, isoniazid or nitrofurantoin).

12. Participants with previous exposure to elafibranor.

13. Participants who are currently participating in, plan to participate in, or have participated in an investigational drug study or medical device study containing active substance within 30 days or 5 half-lives, whichever is longer, prior to the screening period.
i) If the previous study was for an experimental therapy being studied for potential benefit in PBC, and the potential therapeutic agent was proven to have no beneficial effect in PBC and there are no safety concerns, the participant may enrol after 30 days or 5 half lives from the last dose of the therapeutic agent, whichever is longer.
ii) For therapeutic agents being studied for potential benefit in PBC for which it is still unclear if there may be a potential benefit, participants may enrol after 6 months from the last dose of the therapeutic agent.

14. Electrocardiogram (ECG) with QT interval corrected by Fridericia's formula (QTcF)>450 msec in males or QTcF>470 msec in females for participants without bundle branch block. For participants with bundle branch block or other intraventricular conduction delay, a longer QTcF>480 msec would be exclusionary.

15.

$$Total bilirubin ( TB ) > 5 \times ULN .$$

16. Alanine aminotransferase (ALT) and/or aspartate aminotransferase (AST)>5×ULN at SV1:
In cases of ineligibility, the candidate may be subsequently rescreened at investigator's discretion, and enrolled if stable qualifying values are demonstrated.
All AST and ALT values during screening should be analysed via the central laboratory.
For both ALT and AST, the baseline value for the purposes of monitoring for DILI will be the average of any values obtained during screening plus the baseline visit measurement (obtained prior to treatment initiation) but the eligibility of the participant will be based on the screening value(s).

17.

$$Creatinine phosphokinase ( CPK ) > 2 \times ULN .$$

18. Platelet count<50,000/μL.
19. International normalised ratio (INR)>1.8 in the absence of anticoagulant therapy.

20. Estimated glomerular filtration rate (eGFR)<45 mL/min/1.73 m² per the Modification of Diet in Renal Disease (MDRD)-6 Study formula at SV1. In cases of decreased eGFR where the investigator believes the value may not be representative of the actual potential participant's eGFR, retest after adequate hydration is allowed.

21. Significant renal disease, including nephritic syndrome, chronic kidney disease (CKD) (defined as participants with evidence of significantly impaired kidney function or underlying kidney injury).

22. For female participants: known current pregnancy, or has a positive serum pregnancy test, or is breastfeeding.

23. Regular alcohol intake in excess of the recommended limit of 1 standard drink per day for men or women [Crabb 2020 AASLD Practice Guidance], where one standard measure corresponds to 10 g of alcohol.

24. History of alcohol abuse, or other substance abuse within 1 year prior to SV1.

26. Known hypersensitivity to elafibranor or to any of the excipients of the investigational product(s).

27. Mental instability or incompetence, such that the validity of informed consent or ability to be compliant with the study is uncertain.

28. Any other condition that, in the opinion of the investigator, would interfere with study participation or completion, or would put the participant at risk, including a potential participant assessed as being at high risk of noncompliance with the study.

29. Alkaline phosphatase (ALP)≥10×ULN.

30. Albumin<2.8 g/dL due to impaired hepatic function.

### Randomization

Patients who satisfy all eligibility criteria will be randomized in a 1:1 ratio to one of the following groups:
Elafibranor 80 mg/day; or
Placebo.

The randomisation ratio is stratified based on the Child Pugh score at baseline: participants who are Child Pugh A or participants who are Child Pugh B. In addition, the proportion of participants with liver stiffness measurement (LSM)<8 kPa at baseline is capped at 30% of the total study population.

A central randomization system will be used (interactive voice/web response system [IVRS/IWRS]).

### Primary Endpoint

The primary endpoint is event-free survival, defined as the time from randomisation to the first occurrence of any of the following adjudicated clinical outcome events:
All-cause mortality.
Liver transplant.
Liver decompensation, defined as new onset or recurrence of any of the following:
   Variceal bleed;
   Hepatic encephalopathy grade≥2 per West Haven criteria;
   New ascites requiring treatment;
   Refractory ascites or requirement for paracentesis;
Change in MELD 3.0 score to ≥15 in participants with baseline MELD<12, and in whom MELD score change is reflective of worsening liver disease;
Development of hepatocellular carcinoma.

### Secondary Endpoint

The secondary endpoints are:
For the duration of participation in the study:
   Number and percentage of participants who experience TEAEs, treatment related TEAEs, SAEs, and AESIs;
   Number and percentage of participants who develop clinically significant changes from baseline in physical examination findings, vital signs, and ECG;
   Number and percentage of participants who develop clinically significant changes from baseline in haematology, chemistry, liver tests, renal tests (including urinalysis), and other laboratory tests and procedures.
to assess at Month 6, Month 12, and every 12 months up to EOT:
   Change from baseline in ALP;
   Change from baseline in TB;
   Number and percentage of participants with ALP≤1.67×ULN and TB≤ULN;
   Number and percentage of participants with normalisation of TB and ALP, defined as TB<ULN and ALP<ULN;
   Number and percentage of participants with complete biochemical response, defined as normal levels of TB, ALP, transaminases, albumin, and INR;
   Number and percentage of participants with stabilisation in TB (i.e. no increase) defined as TB<1×ULN or increase from baseline<0.1×ULN;
   Number and percentage of participants with a response based on albumin normalization.
to assess at Month 12, and every 12 months up to EOT:
   Change from baseline in LSM assessed by VCTE using Fibroscan®;
   Change from baseline in PBC risk scores: UK PBC score and GLOBE score;
   Number and percentage of participants with LSM≥15 kPa assessed by VCTE using Fibroscan®;
   Number and percentage of participants with no worsening of LSM assessed by VCTE using Fibroscan® defined as no increase of >2 kPa from baseline;
   Change from baseline in AST, ALT, GGT, conjugated bilirubin, albumin, INR and fractionated ALP (hepatic fraction);
   Change from baseline in MELD 3.0 score;
   Change from baseline in Child Pugh grade.
to assess at Month 6, Month 12, and every 12 months up to EOT, as measured by number and percentage of participants with:
   ALP reduction of 40%;
   ALP<1.5×ULN, ALP decrease≥15% and TB≤ULN;
   ALP<1.5×ULN, ALP decrease≥40% and TB≤ULN;
   ALP<1.67×ULN, ALP decrease≥15% and TB≤ULN;
   ALP<3×ULN, AST<2×ULN and TB≤1 mg/dl (Paris I);
   ALP≤1.5×ULN, AST≤1.5×ULN and TB≤1 mg/dl (Paris II criteria);
   Normalisation of abnormal TB;
   Normalisation of abnormal TB and albumin (Rotterdam criteria);
   Reduction in TB to ≤0.6×ULN in participants with TB>0.6×ULN at baseline.
to assess at Month 6, Month 12, and every 12 months up to EOT: Change from baseline in lipid parameters as measured by TC, LDL-C, HDL-C, calculated VLDL-C and TG.
to assess through 6 months up to EOT:
   Change from baseline in PBC Worst Itch NRS score;
   Number and percentage of participants with a response in PBC Worst Itch NRS score defined as ≥2-point reduction from baseline NRS in participants with a baseline NRS≥4;
   Number and percentage of participants with a response in PBC Worst Itch NRS defined as ≥3-point reduction from baseline NRS in participants with a baseline NRS≥4.
to assess at every 6 months up to EOT:
   Change from baseline in 5D-Itch scale;
   Change from baseline in PGI-S;
   Change from baseline in PGI-C;
   Change from baseline in PROMIS Fatigue-Short Form 7a;
   Change from baseline in the ESS;
   Change from baseline in PBC-40 score;
   Change from baseline in EQ-5D-5L;
   Change from baseline in WPAI: GH.
time from randomisation to the first occurrence of each of the following individual adjudicated clinical outcome events:
   All-cause mortality;
   Liver-related mortality;
   Liver transplantation;
   MELD 3.0 score≥15 in participants with baseline MELD score<12;
   Liver decompensation;
   Occurrence of hepatocellular carcinoma;
   Individual PK parameters (during a dosing period of 24 hours) at steady state:
      AUC₀₋₂₄ (area under the plasma concentration-time curve from time 0 to 24 hours);
      Cₘₐₓ (maximum (peak) plasma drug concentration);
      tₘₐₓ (time to reach maximum (peak) plasma concentration following drug administration);
   Population PK parameters:
      CL (apparent clearance of drug from plasma);
      V_{Z} (apparent volume of distribution).

It is expected that elafibranor is better than placebo in preventing clinical outcome events showing disease worsening (including progression of disease leading to liver transplant or death), in participants with PBC and with liver cirrhosis, and more particularly having Child-Pugh score A or having Child-Pugh score B.

1. A method of treating Primary Biliary Cholangitis (PBC) in a subject with liver cirrhosis, said method comprising administering elafibranor, or a pharmaceutically acceptable salt thereof, to the subject.

2. The method according to claim 1, wherein the subject has Child-Pugh score A or has Child-Pugh score B.

3. The method according to claim 1, wherein the subject has Child-Pugh score A.

4. The method according to claim 1, wherein the subject has Child-Pugh score B.

5. The method according to claim 1, wherein the subject has moderate hepatic impairment.

6. The method according to claim 2, wherein the subject is at risk of decompensated cirrhosis.

7. The method according to claim 1, wherein the subject is at risk of liver cirrhosis progression.

8. The method according to claim 1, wherein the subject is at risk of portal hypertension.

9. A method of delaying or preventing liver decompensation in a subject with PBC and with liver cirrhosis, said method comprising administering elafibranor, or a pharmaceutically acceptable salt thereof, to the subject.

10. The method according to claim 9, wherein the subject is at risk of decompensated cirrhosis.

11. The method according to claim 9, wherein the subject has Child-Pugh score A or has Child-Pugh score B.

12. A method of delaying or preventing portal hypertension in a subject with PBC and with liver cirrhosis, said method comprising administering elafibranor, or a pharmaceutically acceptable salt thereof, to the subject.

13. The method according to claim 12, wherein the subject is at risk of portal hypertension.

14. The method according to claim 12, wherein the subject has Child-Pugh score A or has Child-Pugh score B.

15. A method of delaying, preventing, or reducing the risk of liver transplantation in a subject with PBC and with liver cirrhosis, said method comprising administering elafibranor, or a pharmaceutically acceptable salt thereof, to the subject.

16. The method according to claim 15, wherein the subject is at risk of liver transplantation.

17. The method according to claim 15, wherein the subject has Child-Pugh score A or has Child-Pugh score B.

18. The method according to claim 1, wherein the subject responds at least partly to UDCA.

19. The method according to claim 1, wherein the subject has an inadequate response to UDCA.

20. The method according to claim 1, wherein the subject is intolerant to UDCA.

21. The method according to claim 1, for administration at a dose varying from 70 mg to 130 mg, preferably at a dose of 80 mg.

22. The method according to claim 1, wherein the elafibranor, or a pharmaceutically acceptable salt thereof, is in the form of a pharmaceutical composition, preferably an oral pharmaceutical composition.

23. The method according to claim 22, wherein said composition is formulated in the form of injectable suspensions, gels, oils, pills, suppositories, powders, gel caps, capsules, aerosols or means of galenic forms or devices assuring a prolonged and/or slow release.

24. The method according to claim 1, wherein the elafibranor, or a pharmaceutically acceptable salt thereof, is administered orally once daily at a dose of 80 mg.

25. The method according to claim 1, wherein the elafibranor, or a pharmaceutically acceptable salt thereof, is administered in combination with another anti-cholestatic agent, preferably with UDCA.
